(19) United States
(12) Patent Application Publication
(10) Pub. No.: US 2022/0110708 A1
(43) Pub. Date: Apr. 14, 2022

(54) Procedural Barriers with Integrated Medical Device-Connecting Features and Methods Thereof

(71) Applicant: Bard Access Systems, Inc., Salt Lake City, UT (US)

(72) Inventors: Anthony K. Misener, Bountiful, UT (US); Steffan Sowards, Salt Lake City, UT (US)

(21) Appl. No.: 17/499,635

(22) Filed: Oct. 12, 2021

Related U.S. Application Data
(60) Provisional application No. 63/091,142, filed on Oct. 13, 2020.

(51) Int. Cl. A61B 46/23; H01R 31/06; H01R 43/26; H01R 13/516; G02B 6/38; A61B 46/00

(52) U.S. Cl. CPC A61B 46/23; H01R 31/06; H01R 43/26; H01R 2201/12; G02B 6/3825; A61B 46/40; H01R 13/516

(57) ABSTRACT

Disclosed herein are procedural barriers with medical device-connecting features and methods thereof. For example, a procedural barrier can include a sheet of one or more nonwoven materials and an integrated electrical connector, an integrated conduit, or both the integrated electrical connector and the integrated conduit. The integrated electrical connector is integrated into the sheet for establishing an electrical connection across the procedural barrier between a single-use medical device in a procedural field to another medical device on a patient-facing side of the sheet without compromising the procedural field. The integrated conduit passes through the sheet for establishing an alternative electrical connection, an optical connection, or both the alternative electrical connection and the optical connection across the procedural barrier between a same or different single-use medical device in the procedural field to the other medical device on the patient-facing side of the sheet without compromising the procedural field.

### PRIORITY

This application claims the benefit of priority to U.S. Provisional Application No. 63/091,142, filed Oct. 13, 2020, which is incorporated by reference in its entirety into this application.

### BACKGROUND

Procedural fields are typically established about patients with one or more procedural barriers before medical procedures. For example, sterile fields can be established over or around patients by covering the patients with sterile drapes. Oftentimes, the medical procedures require multi-use medical devices that cannot be sterilized, which mandate placement of such medical devices under the sterile drapes; however, sterile single-use medical devices often need to be functionally connected to the multi-use medical devices. Some existing solutions rely on breaching sterile drapes to make functional connections between sterile single-use medical devices and multi-use medical devices. But breaching such sterile drapes risks contaminating the sterile fields carefully established about the patients. That, and establishing some functional connections such as optical connections between the sterile single-use medical devices and the multi-use medical devices can be difficult, particularly through breaches in the sterile drapes. What is needed are procedural barriers with integrated medical device-connecting features that facilitate establishing safe, functional connections between sterile single-use medical devices and multi-use medical devices.

Disclosed herein are procedural barriers with integrated medical device-connecting features and methods thereof that address the foregoing.

### SUMMARY

Disclosed herein is a procedural barrier for a medical procedure. The procedural barrier includes, in some embodiments, a sheet of one or more nonwoven materials and an integrated connecting means in the sheet for connecting one or more single-use medical devices in a procedural field to another medical device on a patient-facing side of the sheet. The sheet has a length and a width sufficient to establish the procedural field for the medical procedure on a side of the sheet opposite the patient-facing side of the sheet. The integrated connection means is selected from an integrated electrical connector integrated into the sheet, an integrated conduit passing through the sheet, and both the integrated electrical connector and the integrated conduit. The integrated electrical connector is integrated into the sheet for establishing an electrical connection across the procedural barrier between a single-use medical device in the procedural field to the other medical device on the patient-facing side of the sheet without compromising the procedural field. The integrated conduit passes through the sheet for establishing an alternative electrical connection, an optical connection, or both the alternative electrical connection and the optical connection across the procedural barrier between a same or different single-use medical device in the procedural field to the other medical device on the patient-facing side of the sheet without compromising the procedural field.

In some embodiments, the procedural barrier includes the integrated electrical connector integrated into the sheet for establishing the electrical connection across the procedural barrier by way of the integrated electrical connector.

In some embodiments, the procedural barrier includes the integrated conduit passing through the sheet adjacent to the integrated electrical connector for establishing the optical connection across the procedural barrier by way of the integrated conduit and the electrical connection across the procedural barrier by way of the integrated electrical connector.

In some embodiments, the procedural barrier further includes a fenestration in the sheet adjacent to the integrated electrical connector. The fenestration is covered by a transparent fenestration cover of a polymeric material having sufficient optical transmissibility for establishing the optical connection across the procedural barrier by way of the fenestration cover without compromising the procedural field.

In some embodiments, the polymeric material is a polyethylene, a polypropylene, or a polyurethane.

In some embodiments, the procedural barrier includes the integrated conduit passing through the sheet for establishing both the alternative electrical connection and the optical connection across the procedural barrier by way of the integrated conduit.

In some embodiments, the sheet includes one or more plies of the one-or-more nonwoven materials. Each ply of the one-or-more plies is formed of a nonwoven material selected from a polypropylene and a wood pulp.

In some embodiments, the sheet includes a single ply of spunbond polypropylene.

In some embodiments, the sheet includes a single ply of spunlace wood pulp.

In some embodiments, the sheet includes one ply of meltblown polypropylene between two plies of spunbond polypropylene.

In some embodiments, the procedural barrier is a sterile procedural drape.

Also disclosed is a method of a procedural barrier for a medical procedure. The method includes, in some embodiments, a procedural barrier-placing step and a device-connecting step. The barrier-placing step includes placing the procedural barrier over a patient. The barrier-placing step establishes a procedural field for the medical procedure on a side of the procedural barrier opposite a patient-facing side of the procedural barrier. The device-connecting step includes connecting one or more single-use medical devices in the procedural field to another medical device on the patient-facing side of the procedural barrier. The procedural barrier includes an integrated connecting means in a sheet of one or more nonwoven materials for the connecting of the one-or-more single-use medical devices to the other medical device selected from an integrated electrical connector integrated into the sheet, an integrated conduit passing through the sheet, or both the integrated electrical connector and the integrated conduit. The integrated electrical connector is integrated into the sheet for establishing an electrical connection across the procedural barrier between a single-use medical device in the procedural field to the other medical device on the patient-facing side of the procedural barrier without compromising the procedural field. The integrated conduit passes through the sheet for establishing an alternative electrical connection, an optical connection, or both the alternative electrical connection and the optical connection across the procedural barrier between a same or different single-use medical device in the procedural field to the other medical device on the patient-facing side of the sheet without compromising the procedural field.

In some embodiments, the device-connecting step includes establishing the electrical connection across the procedural barrier by way of the integrated electrical connector into the sheet.

In some embodiments, the device-connecting step includes establishing the optical connection across the procedural barrier by way of the integrated conduit passing through the sheet adjacent to the integrated electrical connector.

In some embodiments, the device-connecting step includes establishing the optical connection across the procedural barrier by way of a transparent fenestration cover covering a fenestration in the sheet adjacent to the integrated electrical connector. The fenestration cover is of a polymeric material having sufficient optical transmissibility for establishing the optical connection

In some embodiments, the polymeric material is a polyethylene, polypropylene, or polyurethane.

In some embodiments, the device-connecting step includes establishing both the alternative electrical connection and the optical connection across the procedural barrier by way of the integrated conduit passing through the sheet.

In some embodiments, the sheet includes one or more plies of the one-or-more nonwoven materials, each ply of the one-or-more plies formed of a nonwoven material selected from a polypropylene and a wood pulp.

In some embodiments, the sheet includes a single ply of spunbond polypropylene, a single ply of spunlace wood pulp, or one ply of meltblown polypropylene between two plies of spunbond polypropylene.

In some embodiments, the procedural barrier is a sterile procedural drape.

These and other features of the concepts provided herein will become more apparent to those of skill in the art in view of the accompanying drawings and following description, which describe particular embodiments of such concepts in greater detail.

### DRAWINGS

FIG. 1 illustrates a procedural barrier including an integrated electrical connector and a covered fenestration in accordance with some embodiments.

FIG. 2 illustrates a procedural barrier including an integrated electrical connector and integrated conduit in accordance with some embodiments.

FIG. 3 illustrates a procedural barrier including an integrated conduit in accordance with some embodiments.

FIG. 4 illustrates a procedural barrier including a covered fenestration in accordance with some embodiments.

FIG. 5 illustrates a functional connection from a PICC in a sterile field to a relay module of a console outside the sterile field in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

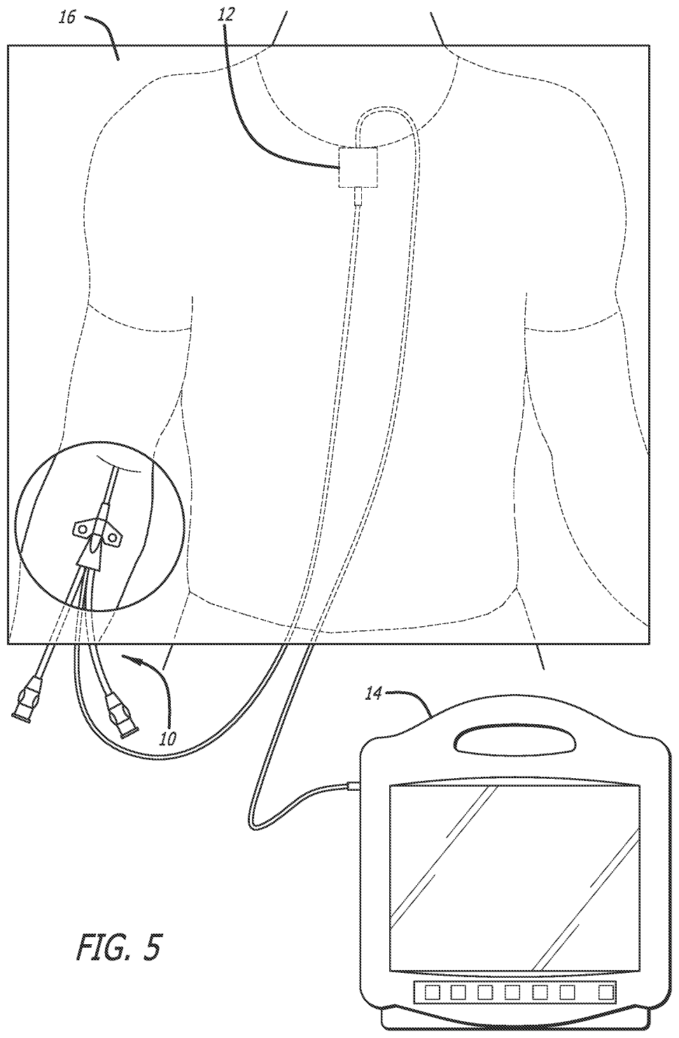

As set forth above, medical procedures often require multi-use medical devices that cannot be sterilized, which mandate placement of such medical devices under sterile drapes where it is difficult to functionally connect them to sterile single-use medical devices in sterile fields over or around patients established by the sterile drapes. As shown in FIG. 5, some existing solutions rely on breaching sterile drapes to make functional connections between sterile single-use medical devices and multi-use medical devices. Indeed, FIG. 5 illustrates a functional connection (e.g. a power-and-data connection) from a peripherally inserted central catheter (“PICC”) 10 in a sterile field to a relay module 12 of a console 14 outside the sterile field by way of a breach in a sterile drape 16. But breaching such sterile drapes risks contaminating the sterile fields carefully established about the patients. That, and establishing some functional connections such as optical connections between the sterile single-use medical devices and the multi-use medical devices can be difficult, particularly through breaches in the sterile drapes.

Disclosed herein are procedural barriers with integrated medical device-connecting features and methods thereof that facilitate establishing safe, functional connections between sterile single-use medical devices and other medical devices such as multi-use medical devices.

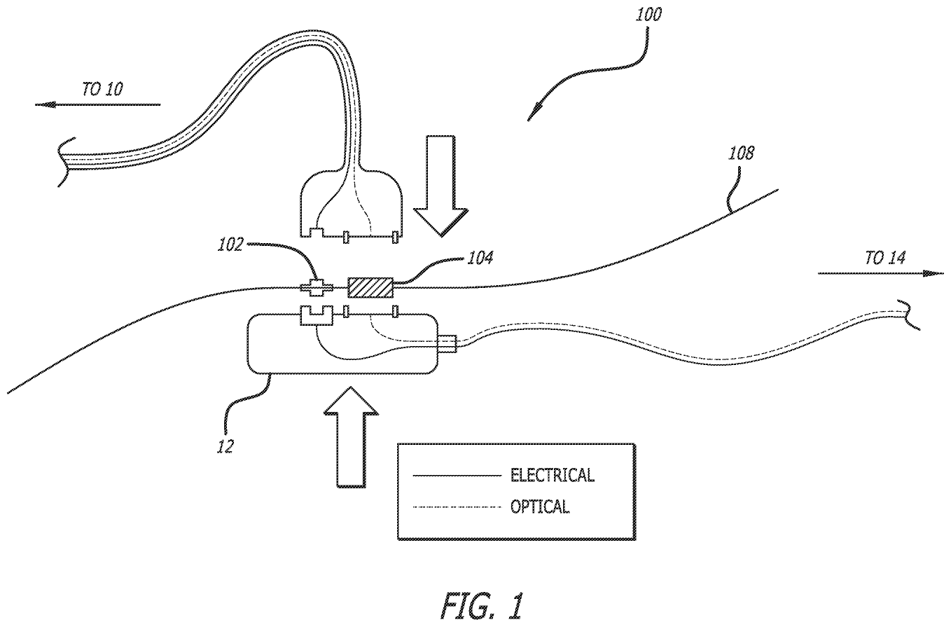

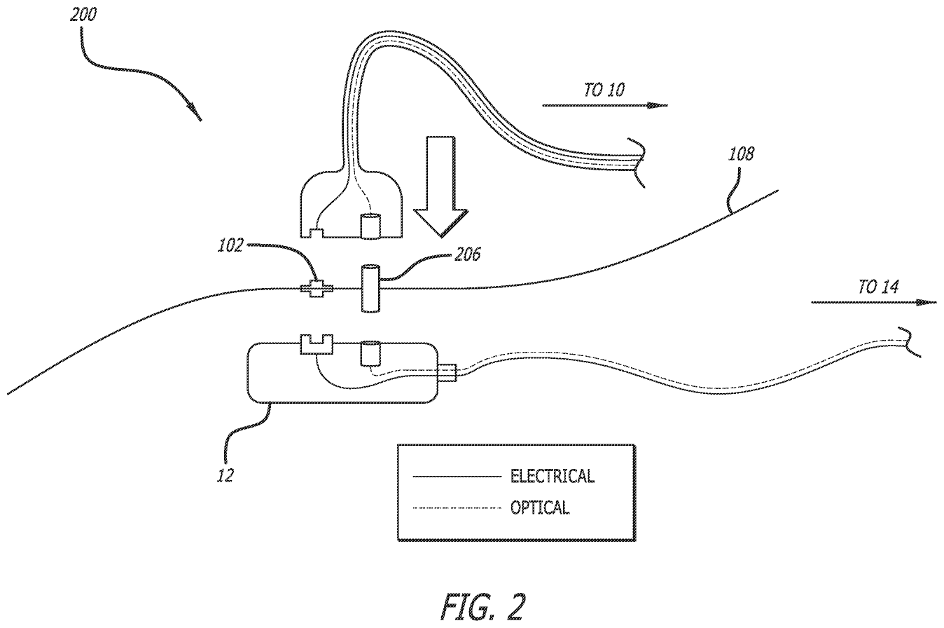

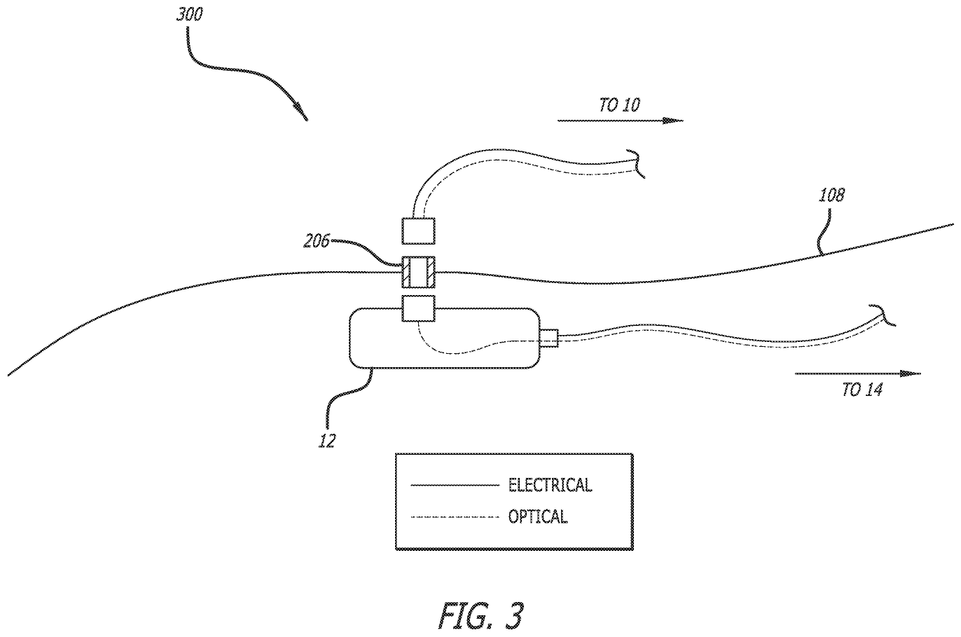

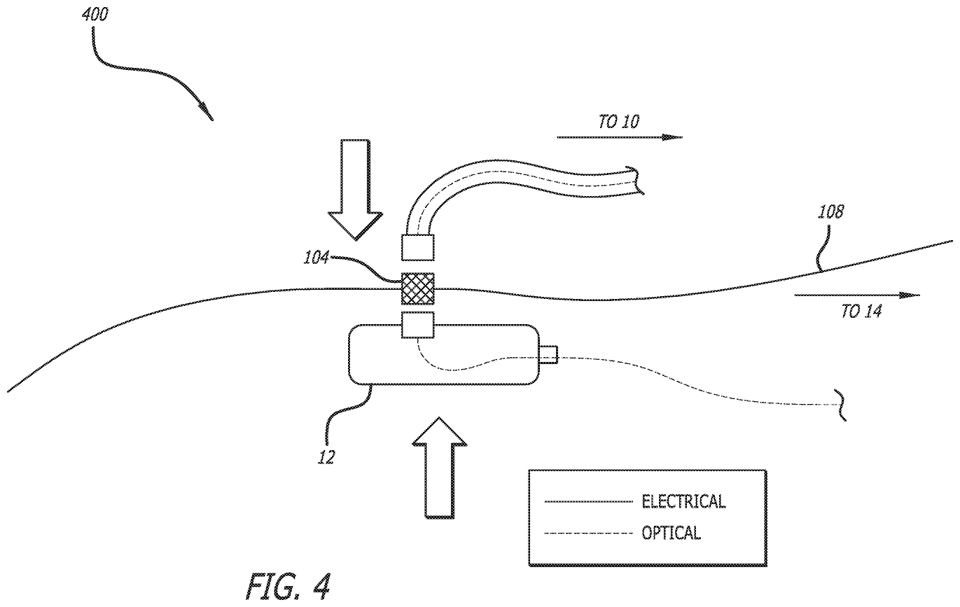

FIG. 1 illustrates a procedural barrier 100 including an integrated electrical 102 connector and a fenestration covered by a fenestration cover 104 in accordance with some embodiments. FIG. 2 illustrates a procedural barrier 200 including the integrated electrical connector 102 and integrated conduit 206 in accordance with some embodiments. FIG. 3 illustrates a procedural barrier 300 including the integrated conduit 260 in accordance with some embodiments. FIG. 4 illustrates a procedural barrier 400 including the fenestration cover 104 in accordance with some embodiments.

Each procedural barrier of the procedural barriers 100, 200, 300, and 400 disclosed herein is for a medical procedure but is not limited thereto. As set forth below, each procedural barrier of the procedural barriers 100, 200, 300, and 400 includes a sheet 108 of one or more nonwoven materials and an integrated connecting means in the sheet 108 for functionally connecting one or more single-use medical devices (e.g. the PICC 10 having an electrocardiogram [“ECG”] stylet, an optical-fiber stylet, or both) in a procedural field over or around a patient to another medical device such as a multi-use medical device (e.g., the console 14 by way of the relay module 12) on a patient-facing side of the procedural barrier 100, 200, 300, or 400 or the sheet 108 thereof (i.e., under the procedural barrier 100, 200, 300, or 400 or sheet 108 thereof). In some embodiments, the procedural barrier 100, 200, 300, or 400 is a sterile drape. In such embodiments, the procedural field is a sterile field.

The sheet 108 has a length and a width sufficient to establish the procedural field for the medical procedure on a side of the sheet 108 opposite the patient-facing side of the sheet 108. Again, such a procedural field can be a sterile field when the procedural barrier 100, 200, 300, or 400 is a sterile drape.

The sheet 108 includes a single ply of the one-or-more nonwoven materials or two or more plies of the one-or-more nonwoven materials. The nonwoven material of any ply of the sheet 108 can be a polypropylene or a wood pulp. In other words, the single ply or each ply of the two-or-more plies of the sheet 108 can independently be a polypropylene or a wood pulp. For example, the sheet 108 can be a single ply of spunbond polypropylene. Alternatively, the sheet 108 can be a single ply of spunlace wood pulp. In another example, the sheet 108 includes one ply of meltblown polypropylene between two plies of spunbond polypropylene.

The integrated connection means is selected from at least an integrated electrical connector 102 integrated into the sheet 108, an integrated conduit 206 passing through the sheet 108, and both the integrated electrical connector 102 and the integrated conduit 206.

As shown in FIGS. 1 and 2, the integrated electrical connector 102 is integrated into the sheet 108 for establishing an electrical connection across the procedural barrier 100 or 200 between a single-use medical device (e.g., the PICC 10 with the ECG stylet) in the procedural field to the multi-use medical device (e.g., the console 14 by way of the relay module 12) on the patient-facing side of the procedural barrier 100 or 200 or the sheet 108 thereof. Notably, the integrated electrical connector 102 enables functional connections such as the foregoing electrical connection without compromising the procedural field. To effectuate the electrical connection, the integrated electrical connector 102 can be a molded piece snapped together over a fenestration in the sheet 108, wherein the molded piece has electrical contacts complementary to both the single-use medical device and the multi-use medical device.

As shown in FIGS. 2 and 3, the integrated conduit 206 passes through the sheet 108 for establishing an alternative electrical connection to that of the integrated electrical connector 102, an optical connection, or both the alternative electrical connection and the optical connection (e.g., a multimodal connection) across the procedural barrier 200 or 300 between a single-use medical device in the procedural field to the multi-use medical device (e.g., the console 14 by way of the relay module 12) on the patient-facing side of the procedural barrier 200 or 300 or the sheet 108 thereof. When both the integrated electrical connector 102 and the integrated conduit 206 are present in the same sheet 108 as shown in FIG. 2 for the procedural barrier 200, the integrated conduit 206 passes through the sheet 108 adjacent to the integrated electrical connector 102 for establishing, for example, an electrical connection across the procedural barrier 200 by way of the integrated electrical connector 102 and an optical connection across the procedural barrier 200 by way of the integrated conduit 206. Notably, the integrated conduit 206 enables functional connections such as the foregoing alternative electrical and optical connections without compromising the procedural field. To effectuate the functional connections, the integrated conduit 206 can be a molded or extruded piece inserted into a fenestration in the sheet 108, wherein the molded or extruded piece has electrical contacts therein complementary to both the single-use medical device and the multi-use medical device, an intermediate optical fiber-containing ferrule therein complementary to both the single-use medical device and the multi-use medical device, or both. Being molded or extruded, the integrated conduit can have alignment feature configured to align the functional connections.

As shown in FIGS. 1 and 4, the procedural barrier 100 and 400 can include a fenestration in the sheet 108 covered by the transparent fenestration cover 104 for establishing an optical connection across the procedural barrier 100 or 400 between a single-use medical device in the procedural field to the multi-use medical device (e.g., the console 14 by way of the relay module 12) on the patient-facing side of the procedural barrier 100 or 400 or the sheet 108 thereof. When both the integrated electrical connector 102 and the fenestration cover 104 are present in the same sheet 108 as shown in FIG. 1 for the procedural barrier 100, the fenestration cover 104 covers a fenestration in the sheet 108 adjacent to the integrated electrical connector 102 for establishing, for example, an electrical connection across the procedural barrier 100 by way of the integrated electrical connector 102 and an optical connection across the procedural barrier 100 by way of the fenestration cover 104. Notably, the fenestration cover 104 enables functional connections such as the foregoing optical connection without compromising the procedural field. To effectuate the optical connection, the fenestration cover 104 can be a transparent polymeric material adhered onto the sheet 108 or between the two-or-more plys of the sheet 108, wherein the fenestration cover 104 has a thickness sufficient for maintaining an integrity of the fenestration cover 104 while establishing the optical connection between, for example, complementarily magnetized optical connectors of the single-use medical device and the multi-use medical device, as well as an optical transmissibility sufficient for transmitting optical signals across the fenestration cover 104.

The fenestration cover 104 includes a single non-laminated transparent sheet of the polymeric material or two-or-more layers of same or different polymeric materials laminated together in a laminated transparent sheet. The polymeric material of the transparent sheet can be a porous, breathable polymeric film such as a polyethylene, a polypropylene, or a polyurethane; however, a porosity of the breathable polymeric film should not be such that optical signals through the fenestration cover 104 are appreciably scattered. For the laminated transparent sheet, each layer of the two-or-more layers can include a same or different polymeric material than an adjacent layer of the two-or-more layers of the laminated transparent sheet.

### Methods

Methods include methods of using a procedural barrier such as the procedural barrier 100, 200, 300, or 400 for a medical procedure. Such a method includes, for example, a procedural barrier-placing step and a device-connecting step.

The barrier-placing step includes placing the procedural barrier 100, 200, 300, or 400 over a patient. The barrier-placing step establishes a procedural field for the medical procedure on a side of the procedural barrier 100, 200, 300, or 400 opposite a patient-facing side of the procedural barrier 100, 200, 300, or 400. As set forth above, the procedural barrier 100, 200, 300, or 400 includes an integrated connecting means in the sheet 108 of the one-or-more nonwoven materials for the connecting of one or more single-use medical devices to another medical device such as a multi-use medical device, wherein the integrated connecting means is selected from the integrated electrical connector 102 integrated into the sheet 108, the integrated conduit 206 passing through the sheet 108, or both the integrated electrical connector 102 and the integrated conduit 206.

The device-connecting step includes connecting the one-or-more single-use medical devices (e.g., the PICC 10) in the procedural field to the multi-use medical device (e.g., the console 14 by way of the relay module 12) on the patient-facing side of the procedural barrier 100, 200, 300, or 400. For example, the device-connecting step includes establishing an electrical connection across the procedural barrier 100 or 200 by way of the integrated electrical connector 102 integrated into the sheet 108 or the integrated conduit 206 passing through the sheet 108. In another example, the device-connecting step includes establishing an optical connection across the procedural barrier 200 or 300 by way of the integrated conduit 206 passing through the sheet 108, optionally together with the electrical connection by way of the integrated conduit 206. In yet another example, the device-connecting step includes establishing the optical connection across the procedural barrier 100 or 400 by way of the transparent fenestration cover 104 covering the fenestration in the sheet 108, optionally along with the electrical connection by way of the integrated electrical connector 102.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A procedural barrier for a medical procedure, comprising:
a sheet of one or more nonwoven materials, the sheet having a length and a width sufficient to establish a procedural field for the medical procedure on a side of the sheet opposite a patient-facing side of the sheet; and
an integrated connecting means in the sheet for connecting one or more single-use medical devices in the procedural field to another medical device on the patient-facing side of the sheet, the integrated connection means selected from:
an integrated electrical connector integrated into the sheet for establishing an electrical connection across the procedural barrier between a single-use medical device in the procedural field to the other medical device on the patient-facing side of the sheet without compromising the procedural field,
an integrated conduit passing through the sheet for establishing an alternative electrical connection, an optical connection, or both the alternative electrical connection and the optical connection across the procedural barrier between a same or different single-use medical device in the procedural field to the other medical device on the patient-facing side of the sheet without compromising the procedural field, and
both the integrated electrical connector and the integrated conduit.

2. The procedural barrier of claim 1, wherein the procedural barrier includes the integrated electrical connector integrated into the sheet for establishing the electrical connection across the procedural barrier by way of the integrated electrical connector.

3. The procedural barrier of claim 2, wherein the procedural barrier includes the integrated conduit passing through the sheet adjacent to the integrated electrical connector for establishing the optical connection across the procedural barrier by way of the integrated conduit and the electrical connection across the procedural barrier by way of the integrated electrical connector.

4. The procedural barrier of claim 2, further comprising a fenestration in the sheet adjacent to the integrated electrical connector, the fenestration covered by a transparent fenestration cover of a polymeric material having sufficient optical transmissibility for establishing the optical connection across the procedural barrier by way of the fenestration cover without compromising the procedural field.

5. The procedural barrier of claim 4, wherein the polymeric material is a polyethylene, a polypropylene, or a polyurethane.

6. The procedural barrier of claim 1, wherein the procedural barrier includes the integrated conduit passing through the sheet for establishing both the alternative electrical connection and the optical connection across the procedural barrier by way of the integrated conduit.

7. The procedural barrier of claim 1, wherein the sheet includes one or more plies of the one-or-more nonwoven materials, each ply of the one-or-more plies formed of a nonwoven material selected from a polypropylene and a wood pulp.

8. The procedural barrier of claim 7, wherein the sheet includes a single ply of spunbond polypropylene.

9. The procedural barrier of claim 7, wherein the sheet includes a single ply of spunlace wood pulp.

10. The procedural barrier of claim 7, wherein the sheet includes one ply of meltblown polypropylene between two plies of spunbond polypropylene.

11. The procedural barrier of claim 1, wherein the procedural barrier is a sterile procedural drape.

12. A method of a procedural barrier for a medical procedure, comprising:
placing the procedural barrier over a patient, thereby establishing a procedural field for the medical procedure on a side of the procedural barrier opposite a patient-facing side of the procedural barrier; and
connecting one or more single-use medical devices in the procedural field to another medical device on the patient-facing side of the procedural barrier, the procedural barrier including an integrated connecting means in a sheet of one or more nonwoven materials for the connecting of the one-or-more single-use medical devices to the other medical device selected from:
an integrated electrical connector integrated into the sheet for establishing an electrical connection across the procedural barrier between a single-use medical device in the procedural field to the other medical device on the patient-facing side of the procedural barrier without compromising the procedural field,
an integrated conduit passing through the sheet for establishing an alternative electrical connection, an optical connection, or both the alternative electrical connection and the optical connection across the procedural barrier between a same or different single-use medical device in the procedural field to the other medical device on the patient-facing side of the sheet without compromising the procedural field, and
both the integrated electrical connector and the integrated conduit.

13. The method of claim 12, wherein connecting the one-or-more single-use medical devices in the procedural field to the other medical device on the patient-facing side of the procedural barrier includes establishing the electrical connection across the procedural barrier by way of the integrated electrical connector into the sheet.

14. The method of claim 13, wherein connecting the one-or-more single-use medical devices in the procedural field to the other medical device on the patient-facing side of the procedural barrier includes establishing the optical connection across the procedural barrier by way of the integrated conduit passing through the sheet adjacent to the integrated electrical connector.

15. The method of claim 13, wherein connecting the one-or-more single-use medical devices in the procedural field to the other medical device on the patient-facing side of the procedural barrier includes establishing the optical connection across the procedural barrier by way of a transparent fenestration cover covering a fenestration in the sheet adjacent to the integrated electrical connector, the fenestration cover of a polymeric material having sufficient optical transmissibility for establishing the optical connection

16. The method of claim 15, wherein the polymeric material is a polyethylene, polypropylene, or polyurethane.

17. The method of claim 12, wherein connecting the one-or-more single-use medical devices in the procedural field to the other medical device on the patient-facing side of the procedural barrier includes establishing both the alternative electrical connection and the optical connection across the procedural barrier by way of the integrated conduit passing through the sheet.

18. The method of claim 12, wherein the sheet includes one or more plies of the one-or-more nonwoven materials, each ply of the one-or-more plies formed of a nonwoven material selected from a polypropylene and a wood pulp.

19. The method of claim 18, wherein the sheet includes a single ply of spunbond polypropylene, a single ply of spunlace wood pulp, or one ply of meltblown polypropylene between two plies of spunbond polypropylene.

20. The method of claim 12, wherein the procedural barrier is a sterile procedural drape.
